(19) United States
(12) Patent Application Publication
(10) Pub. No.: US 2020/0376011 A1
(43) Pub. Date: Dec. 3, 2020

(54) COMPOSITION FOR TREATING CANCER INCLUDING ALGINATE OLIGOSACCHARIDE AS AN ACTIVE INGREDIENT

(71) Applicant: BIO3S, INC, Jeollanam-do (KR)

(72) Inventor: Duwoon KIM, Jeollanam-do (KR)

(73) Assignee: BIO3S, INC

(21) Appl. No.: 16/889,015

(22) Filed: Jun. 1, 2020

Related U.S. Application Data
(60) Provisional application No. 62/856,350, filed on Jun. 3, 2019.

(51) Int. Cl. A61K 31/702; A61P 35/00

(52) U.S. Cl. CPC A61K 31/702; A61P 35/00

(57) ABSTRACT

Disclosed are an anticancer composition comprising an alginate oligosaccharide as an active ingredient and, more specifically, an anticancer composition and an anticancer adjuvant each comprising, as an active ingredient, a mannuronate and guluronate mixed alginate oligosaccharide (AOS) having a higher proportion of mannuronate among alginate oligosaccharides, and a method for preventing or treating cancer by using the composition.

### CROSS-REFERENCE TO RELATED APPLICATIONS

This application claims priority to U.S. Patent Application No. 62/856,350, filed on 3 Jun. 2019. The entire disclosure of the application identified in this paragraph is incorporated herein by reference.

### FIELD

The present disclosure relates to an anticancer composition comprising an alginate oligosaccharide as an active ingredient and, more specifically, to an anticancer composition comprising, as an active ingredient, a mannuronate and guluronate mixed alginate oligosaccharide (hereinafter, AOS) among alginate oligosaccharides and to a method for preventing or treating cancer by using the composition.

### BACKGROUND

Cancer refers to a disease wherein a mass or tumor composed of undifferentiated cells that ignore orders and unlimitedly proliferate in tissues is formed. Ultimately, cancer is the generic term for the group of diseases that can invade and destroy nearby normal tissues or organs and metastasize from a primary site to any organ of an individual to make a new growth site, thereby taking the life of the individual.

The causes of cancer have not yet been revealed, but cancer is considered to be caused by multiple factors including genetic factors as internal factors and cancer-causing factors as external factors, such as carcinogenic chemicals, radiation, ultraviolet and cosmic rays, continuous inflammation and damage, and cancer-causing viral infections. Various causes and unpredictable therapeutic effects render cancer the number one cause of death, and the need for developing medicines therefor is still high.

Approximately 500,000 species of marine organisms, corresponding to about 80% of all species on Earth, are assumed to exist. Out of these, less than 1% has been developed as useful biological sources, and thus marine organisms have a high potential of development. Alginic acid, which is both a marine alga-derived functional material and a representative algal polysaccharide, is contained in 15-30% in brown algae, such as kelp and seaweed, and is characterized by a polyuronide in which two types of uronic acids, β-D-mannuronic acid (M) and α-L-guluronic acid (G), are linked at various ratios via 1,4-glycosidic linkages.

Alginate-derived oligosaccharides may be classified into mannuro-oligosaccharides (MOS), guluro-oligosaccharides (GOS), and mannuronate and guluronate mixed oligosaccharides (alginate oligosaccharide, AOS) according to component sugars, and may also be classified according to the double bond at the end of sugar.

Marine-derived polysaccharides have been used in human life for a long time, and researches on biological activities of marine organism-derived anticancer, antioxidative, antihypertensive, and antibiotic materials are being conducted actively and globally. The production of alginic acid sources produced globally is approximately 100,000 tons, of which about 30% are used as food additives. However, the values of the alginic acid sources can be doubled when the alginic acid sources are developed as high value-added medicinal raw materials. Alginate-derived oligosaccharides have widespread application fields since tangible research achievement has been obtained, for example, biological activities of alginate-derived oligosaccharides according to the structural feature, such as stimulation of root growth of higher plants, acceleration of Bifidobacterium sp. growth, anti-inflammation, antioxidation, and antibiotic activity, have been reported.

Under the circumstances set forth above, the present inventors have conducted intensive researches to develop a preparation capable of effectively treating cancer or effectively slowing down the progression of cancer, and as a result, the present inventors have found that a mannuronate and guluronate mixed oligosaccharide (AOS), which is a type of marine algae oligosaccharides, can efficiently alleviate cancer, and have completed the present disclosure.

### SUMMARY

An aspect of the present disclosure is to provide a pharmaceutical composition comprising an alginate oligosaccharide as an active ingredient for the prevention or treatment of cancer.

Another aspect of the present disclosure is to provide an anticancer adjuvant comprising an alginate oligosaccharide as an active ingredient.

Another aspect of the present disclosure is to provide a method for alleviation, prevention, or treatment of cancer, the method comprising administering to a subject a composition comprising an alginate oligosaccharide as an active ingredient.

In accordance with an aspect of the present disclosure, there is provided a pharmaceutical composition for prevention or treatment of cancer, the pharmaceutical composition comprising an alginate oligosaccharide as an active ingredient.

The alginate oligosaccharide may be a mannuronate and guluronate mixed alginate oligosaccharide (AOS).

The mannuronate and guluronate mixed alginate oligosaccharide may be a non-reducing end unsaturated mannuronate oligosaccharide containing 60% or more of mannuronate and 40% or less of guluronate.

In the pharmaceutical composition, an indicator component of the mannuronate and guluronate mixed alginate oligosaccharide may be a penta-mannuronic acid sodium salt.

In accordance with another aspect of the present disclosure, there is provided an anticancer adjuvant comprising an alginate oligosaccharide as an active ingredient.

The alginate oligosaccharide may be a mannuronate and guluronate mixed alginate oligosaccharide (AOS).

The mannuronate and guluronate mixed alginate oligosaccharide may be a non-reducing end unsaturated mannuronate oligosaccharide containing 60% or more of mannuronate and 40% or less of guluronate.

In the anticancer adjuvant, an indicator component of the mannuronate and guluronate mixed alginate oligosaccharide may be a penta-mannuronic acid sodium salt.

In accordance with still another aspect of the present disclosure, there is provided a method for alleviation, prevention, or treatment of cancer, the method comprising administering to a subject a composition comprising an alginate oligosaccharide as an active ingredient.

The alginate oligosaccharide may be a mannuronate and guluronate mixed alginate oligosaccharide (AOS).

The mannuronate and guluronate mixed alginate oligosaccharide may be a non-reducing end unsaturated mannuronate oligosaccharide containing 60% or more of mannuronate and 40% or less of guluronate.

In the method, an indicator component of the mannuronate and guluronate mixed alginate oligosaccharide may be penta-mannuronic acid sodium salt.

In the present disclosure, alginic acid refers to a compound of molecular formula (C₆H₈O₆)ₙ also called seaweed acid. Alginic acid can be extracted from marine algae, such as kelp. Alginate is known to have generally a wide range of molecular weight of 32-400 kDa. The form of alginate is a fibrous or granular powder, and alginate is usually sold in the form of “sodium alginate” or “potassium alginate”.

In the present disclosure, alginic acid may be obtained by extraction from seaweed, hydrolysis, and then drying. Also, alginic acid is meant to encompass an alginate oligosaccharide, and more specifically, a mannuronate and guluronate mixed alginate oligosaccharide (AOS).

In the present disclosure, the alginate oligosaccharide is a product obtained by enzymatic degradation of alginic acid, and means one composed of a mixture of substances having lower molecular weights when compared with alginic acid. For example, the alginate oligosaccharide may be prepared by hydrolysis of sodium alginate, as a raw material, extracted from natural seaweed, and may be guluronate (G), mannuronate (M), or a glycan in which guluronate (G) and mannuronate (M) are linked via β-1,4 glycosidic linkage.

In the present disclosure, the mannuronate and guluronate mixed alginate oligosaccharide (AOS) may be a non-reducing end unsaturated alginate oligosaccharide having a molecular weight of 30 kDa and containing 60% or more of mannuronate, the mannuronate and guluronate mixed alginate oligosaccharide (AOS) being obtained by the degradation of alginic acid as a substrate through alginate lyase.

In the present disclosure, the mannuronate and guluronate mixed alginate oligosaccharide (AOS) may have a Z-average molecular weight (m/z) of 175 for monosaccharide, 351 for disaccharide, 527 for trisaccharide, 704 for tetrasaccharide, 880 for pentasaccharide, 1056 for hexasaccharide, and 1232 for heptasaccharide.

In the present disclosure, the mannuronate and guluronate mixed alginate oligosaccharide (AOS) may be expressed by the following structural formula.

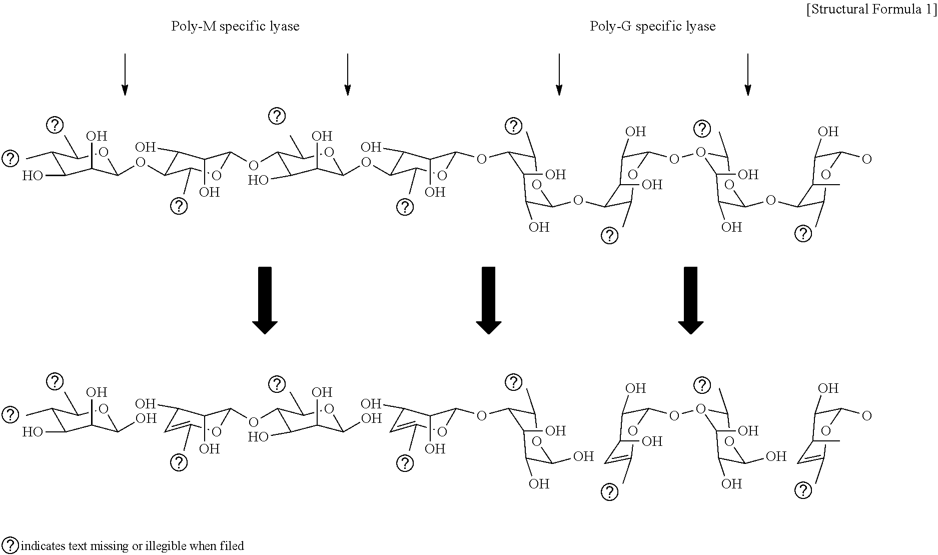

In the present disclosure, substance M5 is an indicator component of the alginate oligosaccharide, and means a penta-mannuronic acid sodium salt.

In the present disclosure, substance M5 may be expressed by the following structure formula.

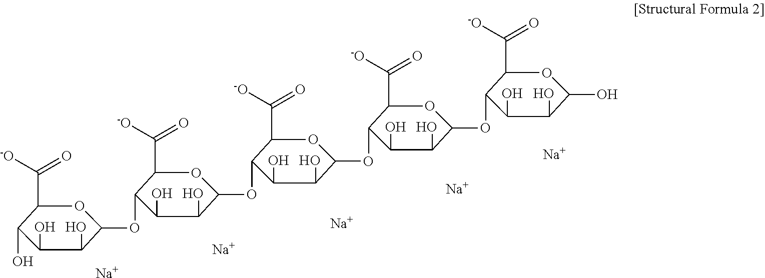

As used herein, the term “non-reducing” refers to a feature of not having anomeric carbon in an oligosaccharide structure, wherein an anomer indicating a type of diastereomerization wherein a hydrogen atom and a hydroxyl group attached on one carbon atom are position-changed with each other in a cyclic reaction resulting in a hemiacetal (forming a ring between C-1 and C-5) and a hemiketal (forming a ring between C-2 and C-5) of a monosaccharide.

As used herein, the term “unsaturated” refers to a form in which a carbon chain with hydrogen atoms is unsaturated, and the term “saturated” refers to a form in which a carbon chain with hydrogen atoms is saturated.

As used herein, the term “non-reducing end unsaturated alginate oligosaccharide” is meant to encompass all types of alginate oligosaccharides in the form in which anomeric carbon is not present in the mannuronate or guluronate at the end of the alginate oligosaccharide and a carbon chain with hydrogen atoms is unsaturated.

As used herein, the term “cancer” refers to a disease that is involved in the regulation of apoptosis and caused by excessive cell proliferation occurring when the normal apoptotic balance is broken. In some cases, these abnormally and excessively proliferating cells infiltrate into nearby tissues or organs to form masses and destroy or change normal structures in the living body. This is called cancer.

In the present disclosure, examples of cancer include cerebrospinal tumor, head and neck cancer, lung cancer, breast cancer, thymoma, esophageal cancer, colon cancer, liver cancer, stomach cancer, pancreatic cancer, biliary cancer, kidney cancer, bladder cancer, prostate cancer, testicular cancer, germ cell tumor, ovarian cancer, cervical cancer, endometrial cancer, lymphoma, acute leukemia, chronic leukemia, multiple myeloma, sarcoma, malignant melanoma, and skin cancer, but the type of cancer in the present disclosure is not limited thereto.

As used herein, the term “anticancer adjuvant” refers to an agent for an auxiliary use in anticancer therapy, and especially a use for promoting the responsiveness of an anticancer drug in the present disclosure.

Therefore, a pharmaceutical composition for an anticancer adjuvant of the present disclosure does not show an anticancer effect by administration thereof alone, but has an auxiliary use for an anticancer drug administered in the anticancer therapy.

The pharmaceutical composition of the present disclosure can be prepared in the form of a pharmaceutical composition further containing an appropriate carrier, excipient, or diluent that is commonly used in the preparation of a pharmaceutical composition.

Specifically, the pharmaceutical composition may be formulated in the form of: oral formulations, such as a powder, granules, a tablet, a capsule, a suspension, an emulsion, syrup, and aerosol; an external preparation; a suppository; and a sterile injectable solution, according to respective common methods.

In the present disclosure, examples of the carrier, excipient, and diluent that may be contained in the pharmaceutical composition may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When the pharmaceutical composition of the present disclosure is formulated into a preparation, an ordinarily used diluent or excipient may be used, such as filler, an extender, a binder, a wetting agent, a disintegrant, or a surfactant.

Examples of a solid preparation for oral administration include a pill, a powder, granules, a capsule, and the like, and such a solid preparation is prepared by mixing the extract and fractions thereof with at least one excipient, for example, starch, calcium carbonate, sucrose or lactose, gelatin, or the like.

In addition, lubricants, such as magnesium stearate and talc, may be used in addition to a simple excipient. A liquid preparation for oral administration corresponds to a suspension, a solution for internal use, an emulsion, syrup, and the like, and examples thereof may include not only simple diluents, such as water and liquid paraffin, but also several excipients, such as a wetting agent, a sweetening agent, a flavoring agent, and a preservative. Examples of a preparation for parenteral administration include a sterile aqueous solution, a non-aqueous solvent, a suspending agent, an emulsion, a lyophilizer, and a suppository. Vegetable oil, such as propylene glycol, polyethylene glycol, or olive oil, and an injectable ester, such as ethyl oleate, may be used as the non-aqueous solvent or the suspending agent. Witepsol, Macrogol, Tween 61, cacao butter, laurin butter, glycerogelatin, or the like may be used as a substrate for the suppository.

The pharmaceutical composition of the present disclosure may be administered in a pharmaceutically effective amount.

As used herein, the term “pharmaceutically effective amount” refers to an amount sufficient to treat or prevent a disease at a reasonable benefit/risk ratio applicable to medical treatment or prevention. The effective dose level may be determined according to: factors including severity of a disease, activity of a drug, a patient's age, body weight, health, and sex, sensitivity of a patient to a drug, administration time, administration route, and excretion rate of the composition of the present disclosure used, duration of treatment, and a drug used simultaneously or in combination with the composition of the present disclosure; and other factors known in the medical field.

The pharmaceutical composition of the present disclosure may be administered alone, or may be administered in combination with a known anticancer agent. Considering all of the factors, it is important to administer the pharmaceutical composition of the present disclosure in an amount at which a maximum effect can be attained by a minimum amount without side effects.

As for the administration route of the anticancer pharmaceutical composition of the present disclosure, the pharmaceutical composition can be administered even through any general route as long as the pharmaceutical composition can arrive at a target tissue. The pharmaceutical composition of the present disclosure may be administered through routes, such as intraperitoneal administration, intravenous administration, intramuscular administration, subcutaneous administration, intradermal administration, oral administration, intranasal administration, and rectal administration, but are not limited thereto, according to the purposes.

As set forth above, the anticancer composition provided in the present disclosure can be utilized as an anticancer drug by having an effect of inhibiting cancer growth, and the naturally-derived substance of the present disclosure can be utilized as an anticancer adjuvant.

### BRIEF DESCRIPTION OF THE DRAWINGS

FIG. 1 illustrates the results of analyzing the molecular weight of a test material according to an example of the present disclosure.

FIG. 2 is a graph of confirming the component ratio of mannuronate (M) and guluronate (G) in an alginate oligosaccharide according to an example of the present disclosure.

FIG. 3 shows the results of confirming a change in tumor size between controls and experimental animals depending on the duration of AOS administration according to an example of the present disclosure.

FIG. 4 shows quantitative analysis of imaging data using bio-luminescence according to an example of the present disclosure. The average tumor size presented for each time is expressed as the luminescence imaging signal intensity.

FIG. 5 is a graph showing an increase in migration of NK cells into ECM of tumor in the co-treatment with the substance of the present disclosure and NK cells according to an example of the present disclosure.

### DETAILED DESCRIPTION

Hereinafter, the present disclosure will be described in detail through examples. However, these examples are given for specifically illustrating the present disclosure, and the scope of the present disclosure is not limited thereto.

### TEST EXAMPLE 1

### Characterization of AOS

The molecular weight of mannuronate and guluronate mixed alginate oligosaccharide (AOS) was analyzed by gel filtration chromatography. The samples were analyzed in a high-performance chromatography system (e2695 Separations Module, Waters, 0.6 mL/min) using a total of four types of columns, Ultrahydrogel 120, 250, 500, and 1000 (1.8×300 nm) while 0.1 M sodium phosphate buffer (pH 7.0) was used as an eluent. Calibration curves were prepared using polyethylene glycol (PEG), and the relative molecular weight was calculated by comparing the retention time between the standard and sample. The molecular weight of sodium alginate as a starting material before degradation was analyzed by gel permeation chromatography analysis. The samples were analyzed in a high-performance chromatography system (HPLC-YL9100, Young Lin instruments, 0.6 mL/min) using Shodex SB-804 HQ and SB-802.5 HQ OHPak columns (Showa Denko, Japan) while 0.1 M sodium phosphate buffer (pH 7.0) was used as an eluent. Calibration curves were prepared using the Pullulan Kit (P-82) and maltooligosaccharides, and the sizes of G2 to G7 and the sizes of monosaccharides and polysaccharides were calculated. The relative molecular weight was calculated by comparing the retention time between the standard and sample.

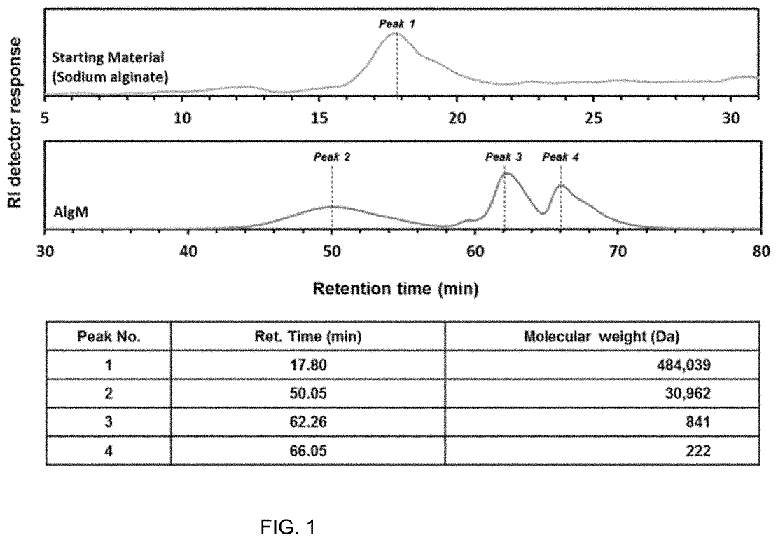

As shown in FIG. 1, the weight average molecular weight (Mw) of sodium alginate as a starting material before degradation was measured to be 484,039 Da as a result of GPC analysis. As a result of GPC analysis, the degraded sodium alginate showed three peaks, corresponding to weight average molecular weights (Mw) of 30,962 Da, 841 Da, and 222 Da, respectively.

Circular dichroism (CD) experiments were carried out using a J-1500 spectrometer (JASCO, Tokyo, Japan) to determine the M/G ratio in AOS. The absorbance of AOS dissolved in PBS solution (2.5 mg/mL) was measured at 190-260 nm and 25° C. using a 1-mm path cell. Specific conditions were as follows: bandwidth, 1 nm; time constant, 1 s; and scanning speed, 200 nm/min. Three spectra corrected for the background were averaged for each sample. The CD spectra of the fermented liquid showed a peak at 201 nm and a trough at 214 nm. The M/G ratio was determined to be 1.97 on the basis of mdeg values, and the AOS was verified to be composed of 66.29% of mannuronate and 33.71% of guluronate.

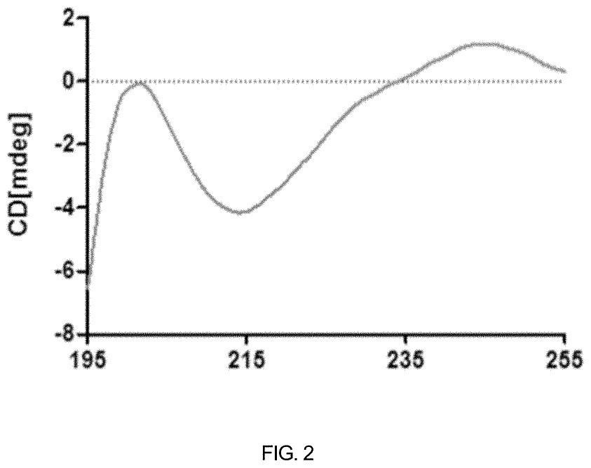

As shown in FIG. 2, the unsaturated alginate oligosaccharide (AOS) was observed to have a characteristic peak from 234 nm. This can be explained by the properties of unsaturated alginate decomposed by alginate lyase.

The indicator substance of AOS was identified to be a penta-mannuronic acid sodium salt, which was named M5.

### TEST EXAMPLE 2

### Preparation of Animal Models

Mice to be tested were provided with usual light cycle and room temperature (22° C.) conditions, and were free to access water and food. Female mice (body weight: 16-22 g, 6-8 week age) without other infections were included in experiments.

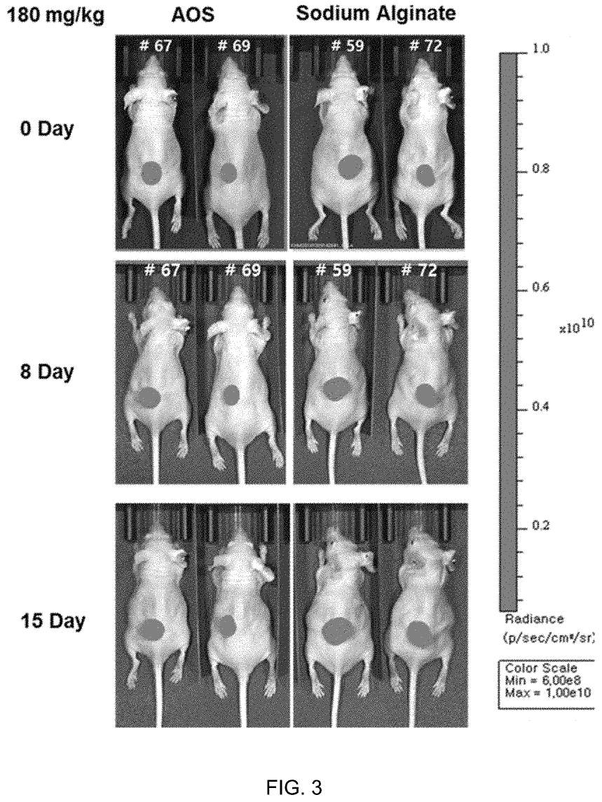

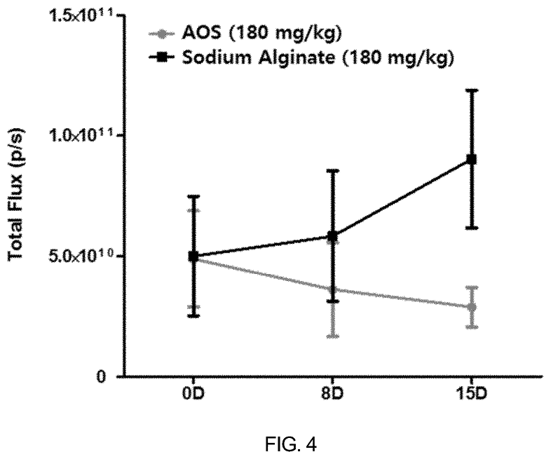

For tumor induction, the female Balb/C nude mice were subcutaneously injected with a minimum oncogenic dose of 1×10⁶ A549 RedFLuc cells (Perkin Elmer) suspended in 50 μl of PBS. As shown in FIGS. 3 and 4, tumor growth was induced until tumor sizes exhibit 5.0×10¹⁰ p/s in total flux (Day 0).

### TEST EXAMPLE 3

### Tumor Size Observation

For the observation of the tumorigenesis inhibitory effect of the mannuronate and guluronate mixed alginate oligosaccharide (AOS), the tumor-induced nude mice were administered with AOS and sodium alginate as a control substance. Administration was conducted five times per week at a dose level of 180 mg/kg/day for each, and observation was twice conducted on Day 8 and Day 15 for luciferase expression. The amount of luciferin injected was 150 mg/kg, and observation was conducted at the time point of 15 min after the injection.

As shown in FIGS. 3 and 4, the tumor size was suppressed from Day 8 after AOS administration, and the average tumor size on Day 15 was 2.88×10¹⁰ p/s in total flux, which was decreased compared with the measurement value on Day 0.

For sodium alginate used as a control, the tumor size on Day 15 was 9.04×10¹⁰ p/s in total flux, indicating that the tumor size was gradually increased. Consequently, AOS was observed to reduce the tumor size by a significant difference when compared with the control.

### TEST EXAMPLE 4

### Verification on Efficacy as Anticancer Adjuvant

The efficacy as a cancer adjuvant was examined by administering tumor microenvironment (TME) simulating models (organ-on-a-chips) with NK cells and the indicator component M5 for the active ingredient of the present disclosure.

NK cells, known to have anti-cancer efficacy as immune cells, were examined for a change in infiltration ability into tumor tissues according to whether or not NK cells were co-administered with M5. The qualification criterion was the infiltration ability of immune cells into ECM, which was determined by the infiltration distance of NK cells and whether or not the number of infiltrating cells was increased. Collagen was selected as a representative type of ECM. The cells were seeded into the chips under a low-concentration collagen condition, and after 6 hours and 48 hours, the infiltration of NK cells toward the medium and cancer cell (A549) channels was observed. Whereas, the infiltration of NK cells was not observed in high-concentration collagen, indicating that the infiltration of immune cells depends on the concentration (intensity) of ECM.

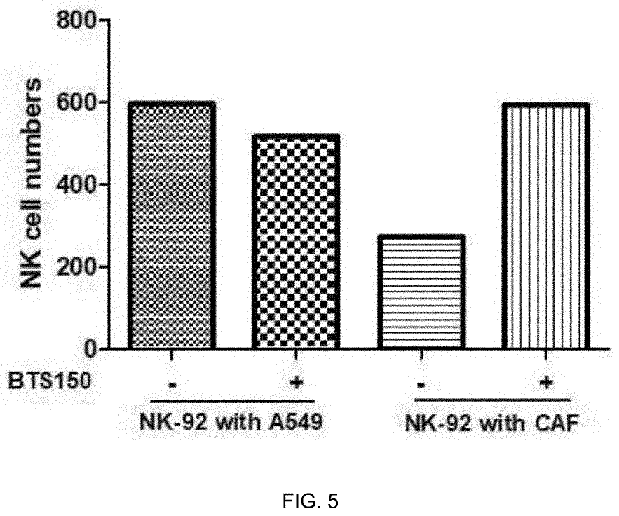

A comparison was made for the infiltration ability of NK cells between the treatment with NK cells alone and the co-treatment with NK cells and M5, for 48 hours in high-concentration collagen cell barrier models. The results are shown in FIG. 5.

As can be seen from FIG. 5, the infiltration ability of the anticancer substance NK cells into cancer cells was enhanced in the co-treatment with NK cells and M5, confirming the effectiveness of the present substance as an anticancer adjuvant.

What is claimed is:

1. A pharmaceutical composition for prevention or treatment of cancer, the pharmaceutical composition comprising an alginate oligosaccharide as an active ingredient.

2. The pharmaceutical composition of claim 1, wherein the alginate oligosaccharide is a mannuronate and guluronate mixed alginate oligosaccharide (AOS).

3. The pharmaceutical composition of claim 2, wherein the mannuronate and guluronate mixed alginate oligosaccharide is a non-reducing end unsaturated mannuronate oligosaccharide containing 60% or more of mannuronate and 40% or less of guluronate.

4. The pharmaceutical composition of claim 2, wherein an indicator component of the mannuronate and guluronate mixed alginate oligosaccharide is a penta-mannuronic acid sodium salt.

5. An anticancer adjuvant comprising an alginate oligosaccharide as an active ingredient.

6. The anticancer adjuvant of claim 5, wherein the alginate oligosaccharide is a mannuronate and guluronate mixed alginate oligosaccharide (AOS).

7. The anticancer adjuvant of claim 6, wherein the mannuronate and guluronate mixed alginate oligosaccharide is a non-reducing end unsaturated mannuronate oligosaccharide containing 60% or more of mannuronate and 40% or less of guluronate.

8. The anticancer adjuvant of claim 6, wherein an indicator component of the mannuronate and guluronate mixed alginate oligosaccharide is a penta-mannuronic acid sodium salt.

9. A method for alleviation, prevention, or treatment of cancer, the method comprising administering to a subject a composition comprising an alginate oligosaccharide as an active ingredient.

10. The method of claim 9, wherein the alginate oligosaccharide is a mannuronate and guluronate mixed alginate oligosaccharide (AOS).

11. The anticancer adjuvant of claim 10, wherein the mannuronate and guluronate mixed alginate oligosaccharide is a non-reducing end unsaturated mannuronate oligosaccharide containing 60% or more of mannuronate and 40% or less of guluronate.

12. The method of claim 10, wherein an indicator component of the mannuronate and guluronate mixed alginate oligosaccharide is a penta-mannuronic acid sodium salt.
